(19) United States
(12) Patent Application Publication
(10) Pub. No.: US 2026/0041572 A1
(43) Pub. Date: Feb. 12, 2026

(54) MEDICAL STENT AND STENT DELIVERY DEVICE

(71) Applicant: OLYMPUS MEDICAL SYSTEMS CORP., Tokyo (JP)

(72) Inventors: Keita OZAWA, Hino-shi (JP); Hirofumi ETO, Akiruno-shi (JP)

(21) Appl. No.: 18/988,741

(22) Filed: Dec. 19, 2024

(30) Foreign Application Priority Data
Aug. 7, 2024 (JP) 2024-130714

(51) Int. Cl. A61F 2/95; A61F 2/82

(52) U.S. Cl. CPC A61F 2/95; A61F 2/82

(57) ABSTRACT

A medical stent whose distal end position can be easily checked under X-ray fluoroscopy, and a stent delivery device are provided. The medical stent includes a main body including a tubular member and a reinforcing material that is wound in a spiral shape and embedded between an inner circumferential surface and an outer circumferential surface of the tubular member, a first locking member that includes a first fixed end fixed to a distal end side of the main body and a first free end located on an outer side from the tubular member, and extends from the first fixed end to the first free end, and a second locking member that includes a second fixed end fixed to a proximal end side of the main body and a second free end located on the outer side from the tubular member, and extends from the second fixed end to the second free end, wherein the main body includes a first region, a second region, and a third region that have different winding pitches of the reinforcing material different from one another, the first region is a region located between the distal end of the main body and the first fixed end, the second region is a region in which the reinforcing material is wound with a greater winding pitch than the first region, and the third region is a region in which the reinforcing material is wound with a greater winding pitch than the second region.

### BACKGROUND OF THE INVENTION

### Field of Invention

The present invention relates to a medical stent and a stent delivery device. Priority is claimed on Japanese Patent Application No. 2024-130714, filed August 7, 2024, the content of which is incorporated herein by reference.

### Description of Related Art

In the related art, a stent that is placed in a bile duct for the purpose of treating stenosis of the bile duct is known (see, for example, Patent Document 1). The stent placed in the bile duct is disposed along the bile duct to maintain a space within the bile duct.

Stents in the related art, such as the stent described in Patent Document 1, are constructed by embedding a coiled wire in a resin body to improve rigidity and flexibility.

When a stent is placed in a bile duct, a surgeon checks the position of the stent by confirming a metal wire under X-ray fluoroscopy. Thus, the surgeon can dispose the stent at a predetermined position in the bile duct.

### [Patent Documents]

[Patent Document 1] Japanese Patent No. 6995219

### SUMMARY OF THE INVENTION

However, in a stent placed in a bile duct, when a wire is not provided at a distal end of the stent, there is a possibility that a surgeon may not be able to accurately check the position of the distal end of the stent under X-ray fluoroscopy.

In light of the above circumstances, an object of the present invention is to provide a medical stent and a stent delivery device in which a distal end position can be easily checked under X-ray fluoroscopy.

In order to solve the above problems, the present invention proposes the following means.

A medical stent of the present invention includes: a main body including a tubular member that has a tubular shape and extends in a longitudinal direction thereof, and a reinforcing material that is wound in a spiral shape around a longitudinal axis extending in the longitudinal direction to extend in the longitudinal direction and embedded between an inner circumferential surface of the tubular member and an outer circumferential surface of the tubular member; a first locking member that includes a first fixed end fixed to a distal end side in the longitudinal direction of the main body and a first free end located on an outer side in a radial direction of the main body from the outer circumferential surface of the tubular member, and extends from the first fixed end to the first free end toward a proximal end side in the longitudinal direction; and a second locking member that includes a second fixed end fixed to the proximal end side of the main body and a second free end located on the outer side from the outer circumferential surface of the tubular member, and extends from the second fixed end to the second free end toward the distal end side. The main body includes a first region, a second region, and a third region that have different winding pitches of the reinforcing material from one another, the first region is a region located between the distal end of the main body and the first fixed end in the longitudinal direction, the second region is a region that is located on the proximal end side of the first region, and in which the reinforcing material is wound in a spiral shape with a greater winding pitch than the first region, and the third region is a region that is located on the proximal end side of the second region, and in which the reinforcing material is wound in a spiral shape with a greater winding pitch than the second region.

A stent delivery device of the present invention includes: a guide catheter including a guide tube through which a guide wire is configured to be inserted, and a wire connected to the guide tube; a pusher catheter including a first tube that includes a first lumen through which the wire is configured to be inserted, and a second tube that is connected to a distal end part of the first lumen and includes a second lumen through which the guide tube is configured to be inserted; a coupling member that includes a loop through which the guide tube is configured to be inserted and is coupled to the distal end part of the pusher catheter; and a stent that extends in a longitudinal direction thereof and is located on a distal end side in the longitudinal direction from a distal end of the second tube. The stent includes: a main body including a tubular member having a tubular shape, which extends in the longitudinal direction and through which the guide tube is inserted, a reinforcing material that is wound in a spiral shape around a longitudinal axis extending in the longitudinal direction to extend in the longitudinal direction and embedded between an inner circumferential surface of the tubular member and an outer circumferential surface of the tubular member, and a through hole that penetrates the tubular member from the inner circumferential surface to the outer circumferential surface and is provided on a proximal end side in the longitudinal direction; a first locking member that includes a first fixed end fixed to the distal end side of the main body and a first free end located on an outer side in a radial direction of the main body from the outer circumferential surface of the tubular member, and extends from the first fixed end to the first free end toward the proximal end side; and a second locking member that includes a second fixed end fixed to the proximal end side of the main body and a second free end located on the outer side from the outer circumferential surface of the tubular member, and extends from the second fixed end to the second free end toward the distal end side. The main body includes a first region, a second region, and a third region that have different winding pitches of the reinforcing material from one another, the first region is a region located between the distal end of the main body and the first fixed end in the longitudinal direction, the second region is a region that is located on the proximal end side of the first region, and in which the reinforcing material is wound in a spiral shape with a greater winding pitch than the first region, and the third region is a region that is located on the proximal end side of the second region, and in which the reinforcing material is wound in a spiral shape with a greater winding pitch than the second region. The loop of the coupling member is inserted into the through hole, and the guide tube is inserted into the loop, thereby positioning the stent relative to the pusher catheter.

According to the medical stent and the stent delivery device of the present invention, it is possible to provide a medical stent and a stent delivery device in which a distal end position can be easily checked under X-ray fluoroscopy.

### BRIEF DESCRIPTION OF THE DRAWINGS

FIG. 1 is an overall view of a stent delivery device according to the present embodiment.

FIG. 2 is a side view of a stent according to the present embodiment.

FIG. 3 is a perspective view of the stent.

FIG. 4 is a schematic cross-sectional view of the stent delivery device.

FIG. 5 is a diagram showing a process of placing the stent using the stent delivery device.

FIG. 6 is a diagram showing the placed stent.

FIG. 7 is a diagram showing a process of pulling the stent into a channel of an endoscope.

### DETAILED DESCRIPTION OF THE INVENTION

A stent delivery device 1 and a medical stent 10 according to embodiments of the present invention will be described with reference to the drawings.

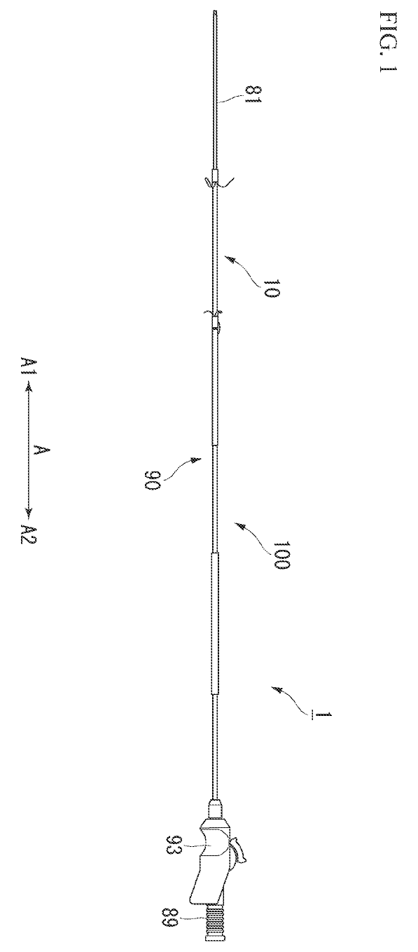

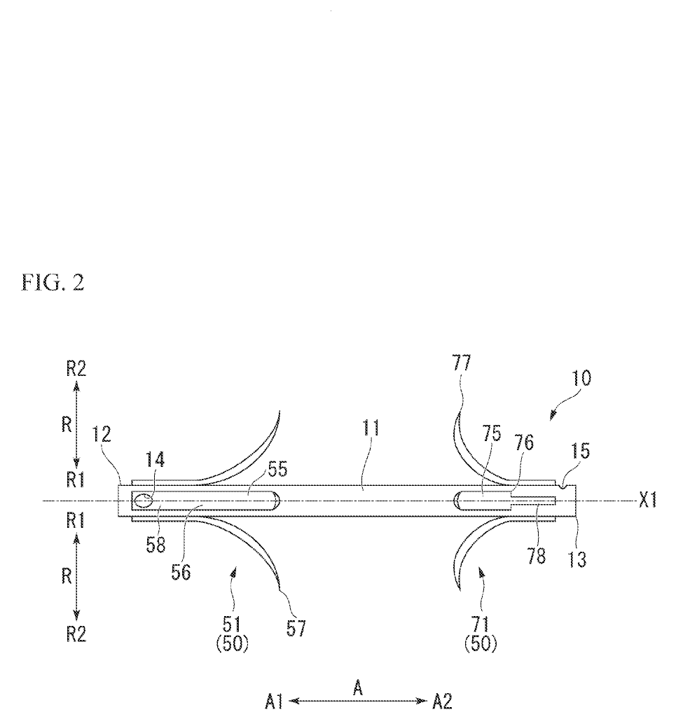

FIG. 1 is an overall view of a stent delivery device 1 according to the present embodiment. FIG. 2 is a side view of a stent 10.

The stent delivery device 1 includes a medical stent (stent) 10 and a delivery catheter 100.

The stent 10 is a stent placed in a bile duct, and includes a tubular main body 11 and flaps 50 attached to both end parts of the main body 11.

The main body 11 is a tubular member that has a distal end 12 and a proximal end 13, and extends along a longitudinal axis X1. The distal end 12 is an end that is disposed on a liver side when placed in the bile duct. The proximal end 13 is an end that is disposed on a duodenal papilla side when placed in the bile duct.

In the following description, a direction in which the longitudinal axis X1 extends will be referred to as a “longitudinal direction A.” In addition, in the stent 10, a side on which the distal end 12 of the main body 11 is provided will be referred to as a “distal end side (distal end side) A1” in the longitudinal direction A, and a side on which the proximal end 13 is provided will be referred to as a “proximal end side (proximal end side) A2” in the longitudinal direction A.

Further, in the stent 10, a direction perpendicular to the longitudinal direction A will be referred to as a “radial direction R,” a side closer to the longitudinal axis X1 will be referred to as an “inner side R1” in the radial direction R, and a side away from the longitudinal axis X1 will be referred to as an “outer side R2” in the radial direction R.

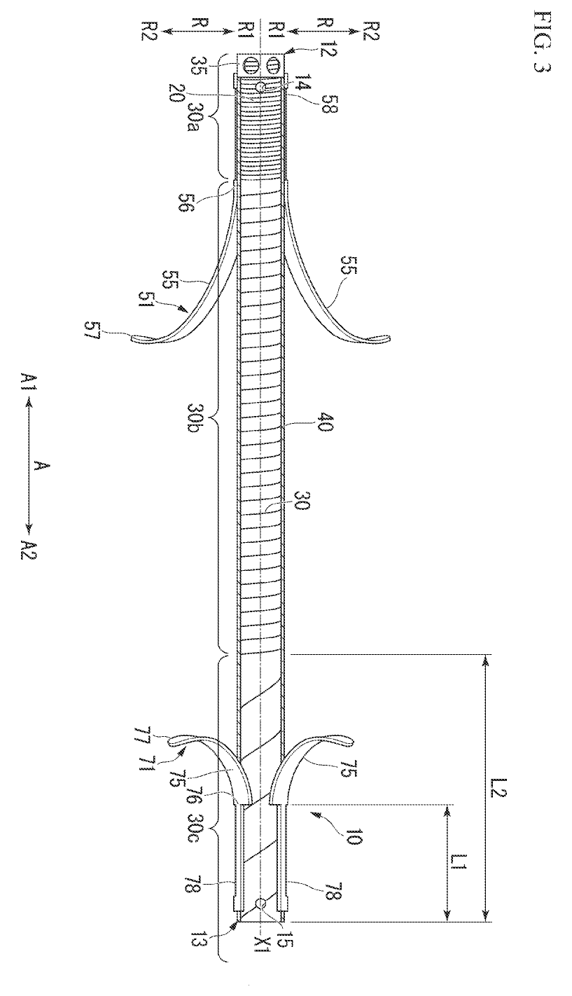

FIG. 3 is a perspective view of the stent 10, with an outer layer of the stent 10 shown transparently.

The main body 11 has a resin inner layer 20, a metal wire (reinforcing material) 30 that is wound around the inner layer 20, and a resin outer layer 40 that covers the inner layer 20 and the wire 30.

The inner layer 20 and the outer layer 40 form a “tubular member” that has a tubular shape and extends in the longitudinal direction A of the main body 11.

The wire 30 is embedded between an inner circumferential surface and an outer circumferential surface of the tubular member in the main body 11.

The main body 11 has, for example, the same outer diameter from the distal end 12 to the proximal end 13.

The inner layer 20 is a tube made of a resin material that forms a smooth surface and has biocompatibility, such as polytetrafluoroethylene (PTFE) or perfluoroalkoxy alkane (PFA).

The wire 30 is a coil-shaped member wound in a spiral shape around the longitudinal axis X1 on an outer circumferential surface of the inner layer 20. By winding the wire 30 in a spiral shape between the inner circumferential surface and the outer circumferential surface of the main body 11, buckling of the stent 10 can be inhibited, and flexibility of the stent 10 can be improved.

The material of the wire 30 is, for example, a material that is opaque to X-rays, such as a tungsten steel or a stainless steel.

The main body 11 has a plurality of regions that have different winding pitches of the wire 30 from one another. Details of the plurality of regions of the main body 11 will be described later.

The outer layer 40 is made of a resin material that has elasticity, flexibility, and biocompatibility, such as urethane or polyethylene. The outer layer 40 is also provided in each gap between adjacent wires 30 in the longitudinal direction A.

The flaps 50 have a first flap 51 provided on the distal end side A1, and a second flap 71 provided on the proximal end side A2.

The first flap 51 has a plurality of first locking members 55. The first locking member 55 has a first fixed end 56 and a first free end 57, and is an elongated member extending between the first fixed end 56 and the first free end 57.

In the first locking member 55, a rib (joint part) 58 extends from the first fixed end 56 to the distal end side A1. In the present embodiment, the width of the rib 58 is the same as the width of the first locking member 55.

The first locking member 55 is attached to the main body 11 by joining the rib 58 to the outer circumferential surface of the main body 11. The rib 58 is joined to the main body 11 using a joining method such as heat fusion or adhesion.

The first fixed end 56 of the first locking member 55 is fixed to the main body 11 by joining the rib 58 to the main body 11. In the first flap 51 connected to the main body 11, the first fixed end 56 is a boundary part between a portion that is joined to the main body 11 by heat fusion, adhesion, or the like and a portion that is not joined to the main body 11.

The second flap 71 has a plurality of second locking members 75. The second locking member 75 has a second fixed end 76 and a second free end 77, and is an elongated member extending between the second fixed end 76 and the second free end 77.

In the second locking member 75, a rib (joint part) 78 extends from the second fixed end 76 to the proximal end side A2. In the present embodiment, the width of the rib 78 is smaller than the width of the second locking member 75.

The second locking member 75 is attached to the main body 11 by joining the rib 78 to the outer circumferential surface of the main body 11. The rib 78 is joined to the main body 11 using a joining method such as heat fusion or adhesion.

The second fixed end 76 of the second locking member 75 is fixed to the main body 11 by joining the rib 78 to the main body 11. In the second flap 71 connected to the main body 11, the second fixed end 76 is a boundary between a portion that is joined to the main body 11 by heat fusion, adhesion, or the like and a portion that is not joined to the main body 11.

The second free end 77 is located on the outer side R2 of the outer circumferential surface of the main body 11. The second locking member 75 extends from the second fixed end 76 to the second free end 77 toward the distal end side A1 and the outer side R2. The second locking member 75 gradually extends toward the distal end side A1 and the outer side R2.

In the present embodiment, a length of the second locking member 75 in the longitudinal direction A is shorter than a length of the first locking member 55 in the longitudinal direction A.

The number of the first locking members 55 in the first flap 51 and the number of the second locking members 75 in the second flap 71 can be set appropriately. In the present embodiment, the first flap 51 has three first locking members 55, and the second flap 71 has three second locking members 75. The three first locking members 55 and the three second locking members 75 are attached at equal intervals around the longitudinal axis X1 on the outer circumferential surface of the main body 11.

Phases on the outer circumferential surface of the main body 11 to which the first locking members 55 and the second locking members 75 are attached in the first flap 51 and the second flap 71 may be the same or different from one another. The number of the first locking members 55 and the number of the second locking members 75 in the first flap 51 and the second flap 71 may be the same or different from each other.

The numbers of the ribs 58 and 78 in the first and second flaps 51 and 71 may be different from the numbers of the first and second locking members 55 and 75 in the first and second flaps 51 and 71.

For example, the first flap 51 and the second flap 71 may have the first locking members 55 and the second locking members 75 to which the ribs 58 and 78 are not connected. In this case, the first locking members 55 and the second locking members 75 to which the ribs 58 and 78 are not connected are fixed to the main body 11 by joining roots (the first fixed ends 56 and the second fixed ends 76) of the first locking members 55 and the second locking members 75 to the outer circumferential surface of the main body 11.

The main body 11 has a first region 30a, a second region 30b, and a third region 30c that have different winding pitches of the wire 30 from one another. The winding pitch of the wire 30 in the first region 30a is substantially uniform, the winding pitch of the wire 30 in the second region 30b is substantially uniform, and the winding pitch of the wire 30 in the third region 30c is substantially uniform.

The first region 30a is a region that is located between the distal end 12 of the main body 11 and the first fixed end 56 in the longitudinal direction A. In the present embodiment, a distal end of the first region 30a is located at the distal end 12 of the main body 11.

The first region 30a is disposed between the distal end 12 of the main body 11 and the first fixed end 56, and thus, for example, when the stent 10 is placed in the bile duct, the position of the distal end 12 of the main body 11 can be confirmed by visually observing the distal end of the first region 30a under X-ray fluoroscopy.

The second region 30b is adjacent to the first region 30a in the longitudinal direction A and is provided on the proximal end side A2 of the first region 30a.

The second region 30b is a region in which the number of turns of winding the wire 30 per unit length is fewer than that of the first region 30a. That is, the second region 30b is a region in which the wire 30 is wound in a spiral shape with a greater winding pitch than the first region 30a.

The third region 30c is adjacent to the second region 30b in the longitudinal direction A and is provided on the proximal end side A2 of the second region 30b.

The third region 30c is a region in which the number of turns of winding the wire 30 per unit length is smaller than that of the second region 30b. That is, the third region 30c is a region in which the wire 30 is wound in a spiral shape with a greater winding pitch than the second region 30b.

For that reason, among the first region 30a, the second region 30b, and the third region 30c in the main body 11, the first region 30a has the greatest rigidity and is less likely to be deformed than the second region 30b and the third region 30c against forces applied in the longitudinal direction A and the radial direction R.

In terms of a winding angle (inclination) of the wire 30 with respect to the longitudinal axis X1, a winding angle of the wire 30 in the first region 30a is closer to a right angle than a winding angle of the wire 30 in the second region 30b.

The winding angle of the wire 30 in the second region 30b is closer to a right angle than a winding angle of the wire 30 in the third region 30c.

A boundary between the first region 30a and the second region 30b is provided at the same position as the first fixed end 56 in the longitudinal direction A. The boundary between the first region 30a and the second region 30b does not have to be strictly at the same position as the first fixed end 56.

Here, as shown in FIG. 3, the distance in the longitudinal direction A from the second fixed end 76 to the proximal end 13 of the main body 11 will be referred to as a first distance L1. Also, the distance in the longitudinal direction A from a boundary between the second region 30b and the third region 30c to the proximal end 13 of the main body 11 will be referred to as a second distance L2.

The second distance L2 is at least twice the first distance L1. By making the second distance L2 at least twice the first distance L1, the distance in the longitudinal direction A from the boundary between the second region 30b and the third region 30c to the proximal end 13 of the main body 11 can be sufficiently maintained, and when the stent 10 is pulled into a channel of an endoscope, the third region 30c having a smaller rigidity can be reliably folded back and pulled into the channel. A process of pulling the stent 10 into the channel of the endoscope will be described later.

In the present embodiment, a proximal end of the third region 30c is located at the proximal end 13 of the main body 11.

In the longitudinal direction A, the boundary between the second region 30b and the third region 30c is preferably located at a center of the stent 10. The boundary between the second region 30b and the third region 30c is disposed at the center of the stent 10, and thus, for example, when the stent 10 is placed in the bile duct, the center position of the stent 10 can be confirmed by visually observing the boundary between the second region 30b and the third region 30c under X-ray fluoroscopy.

The wire 30 is fixed to the main body 11 by a metal ring 35. The ring 35 is an annular member with the longitudinal axis X1 set as its central axis, and is provided at the distal end of the first region 30a.

If the wire 30 can be sufficiently fixed to the main body 11 without the ring 35, the stent 10 does not need to include the ring 35.

A first through hole (through hole) 14 and a second through hole (through hole) 15 that penetrate the main body 11 from the inner circumferential surface to the outer circumferential surface are formed in the main body 11.

When the stent 10 is placed in the bile duct, bile can flow from the outer circumferential surface side of the main body 11 into an internal space of the main body 11 through the through holes 14 and 15. The number and positions of the through holes 14 and 15 can be set appropriately. The through holes 14 and 15 may overlap partly or entirely with the rib 58 or the rib 78.

In the present embodiment, the first through hole 14 is provided on the distal end side A1 of the main body 11, and is provided at a position at which it overlaps the first region 30a in the longitudinal direction A. Specifically, the first through hole 14 is provided on the distal end side A1 of the first fixed end 56.

The second through hole 15 is provided on the proximal end side A2 of the main body 11, and is provided at a position at which it overlaps the third region 30c in the longitudinal direction A.

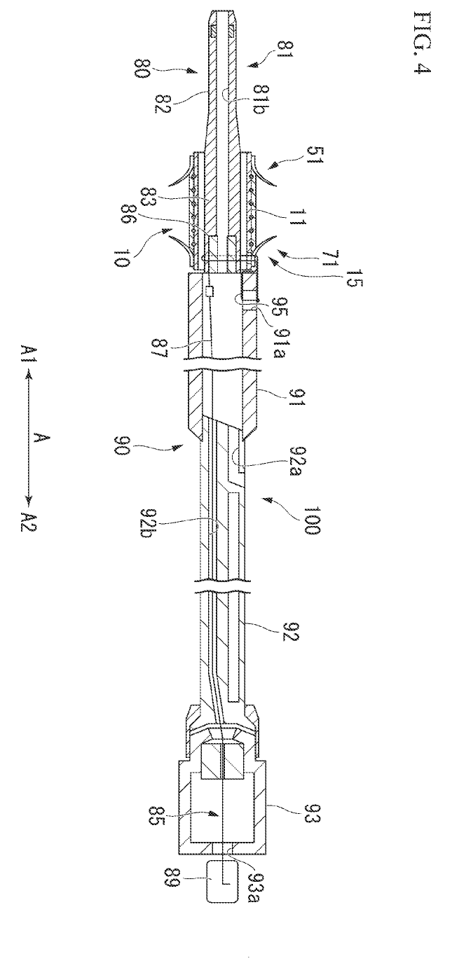

FIG. 4 is a schematic cross-sectional view showing a structure of the stent delivery device 1.

The delivery catheter 100 includes a guide catheter 80 and a pusher catheter 90.

The guide catheter 80 includes a tube (guide tube) 81 through which a guide wire can be inserted, and a traction part 85 for pulling the tube 81 toward the proximal end side A2. The guide wire is an elongated member used when the stent 10 is placed in the bile duct.

The tube 81 is a tubular member made of a resin, and has an inner lumen through which the guide wire can be inserted. The tube 81 is flexible enough to be deformable when the tube 81 comes into contact with biological tissue when the stent delivery device 1 is used.

The tube 81 is an elastic member having a restoring force, and when no external force is applied, the tube 81 becomes straight due to the restoring force. An inner circumferential surface 81b of the tube 81 is circular when viewed in a radial cross-section of the tube 81.

The tube 81 has a thin diameter part 82 located at the distal end side A1 and a thick diameter part 83 located at the proximal end side A2. The outer diameter of the thick diameter part 83 is greater than the outer diameter of the thin diameter part 82.

An outer circumferential surface of the thin diameter part 82 and an outer circumferential surface of the thick diameter part 83 are connected to each other via a smooth curved surface. As a result, the outer diameter of the tube 81 gradually increases from the thin diameter part 82 to the thick diameter part 83.

The outer diameters of the thin diameter part 82 and the thick diameter part 83 are smaller than the inner diameter of the stent 10. Accordingly, the stent 10 can be attached to the tube 81 by inserting the tube 81 into the stent 10.

If the diameter of the stent 10 is small, the outer diameter of the tube 81 may be the same in the longitudinal direction A.

The traction part 85 has a pipe 86, a wire 87, and an operation part 89. The pipe 86 is a metallic tubular member with both axial ends open.

The pipe 86 is embedded in a wall of the tube 81 coaxially with the tube 81. The pipe 86 is provided at a proximal end portion of the thick diameter part 83.

The wire 87 is an elongated metallic member with a distal end welded to the pipe 86 and a proximal end connected to the operation part 89.

The pusher catheter 90 has a single lumen tube (second tube) 91, a multi-lumen tube (first tube) 92, and a gripping part 93.

The single lumen tube 91 is a tubular member having an internal space (second lumen) into which the thick diameter part 83 of the tube 81 can enter. The single lumen tube 91 is flexible.

A surface on the distal end side A1 (distal end surface) of the single lumen tube 91 is a plane perpendicular to a central axis of the single lumen tube 91. The distal end surface of the single lumen tube 91 can abut the proximal end of the stent 10 to support the stent 10.

A thickness of the single lumen tube 91 is greater than a thickness of the main body 11 of the stent 10. The thickness of the single lumen tube 91 refers to a difference in dimension between the outer diameter and the inner diameter of the single lumen tube 91. The thickness of the main body 11 refers to a difference in dimension between the outer diameter and the inner diameter of the main body 11.

The single lumen tube 91 has a length that allows the thick diameter part 83 of the tube 81 to be completely accommodated inside the single lumen tube 91. The material of the single lumen tube 91 is not particularly limited, but is preferably a resin.

The multi-lumen tube 92 is fixed to a proximal end part of the single lumen tube 91. The multi-lumen tube 92 has a guide wire lumen 92a for inserting the guide wire and a wire lumen (first lumen) 92b.

The guide wire lumen 92a opens at a distal end of the multi-lumen tube 92, and opens to a side surface of the multi-lumen tube 92 on the proximal end side A2 from the distal end of the multi-lumen tube 92.

The wire 87 of the guide catheter 80 is inserted into the wire lumen 92b. The wire lumen 92b opens at the distal end and a proximal end of the multi-lumen tube 92.

The gripping part 93 is coupled to the proximal end of the multi-lumen tube 92. The gripping part 93 is a substantially cylindrical member with a greater diameter than the multi-lumen tube 92. Irregularities for preventing slipping or the like may be formed on an outer circumferential surface of the gripping part 93.

A through hole (gripping through hole) 93a that communicates with the wire lumen 92b is formed in the gripping part 93. In the present embodiment, the through hole 93a is located on an extension line formed by extending a central axis of the multi-lumen tube 92 toward the proximal end side A2.

The wire 87 of the guide catheter 80 is inserted through the through hole 93a. The wire 87 and the operation part 89 connected to a proximal end part of the wire 87 extend further toward the proximal end side A2 than the through hole 93a.

A hole 91a that communicates with the internal space (second lumen) of the single lumen tube 91 is provided on an outer circumferential surface at the distal end part of the single lumen tube 91.

A thread (coupling member) 95 is passed through the hole 91a. An end part of the thread 95 is tied to form a loop (annular ring). The looped thread 95 passes through the second through hole 15 from the outer side R2 to the inner side R1 in the main body 11 of the stent 10, and enters an inside of the main body 11.

The tube 81 of the guide catheter 80 is inserted in the longitudinal direction A through the loop of the thread 95 inside the stent 10.

Next, an operation when the stent delivery device 1 is used will be described.

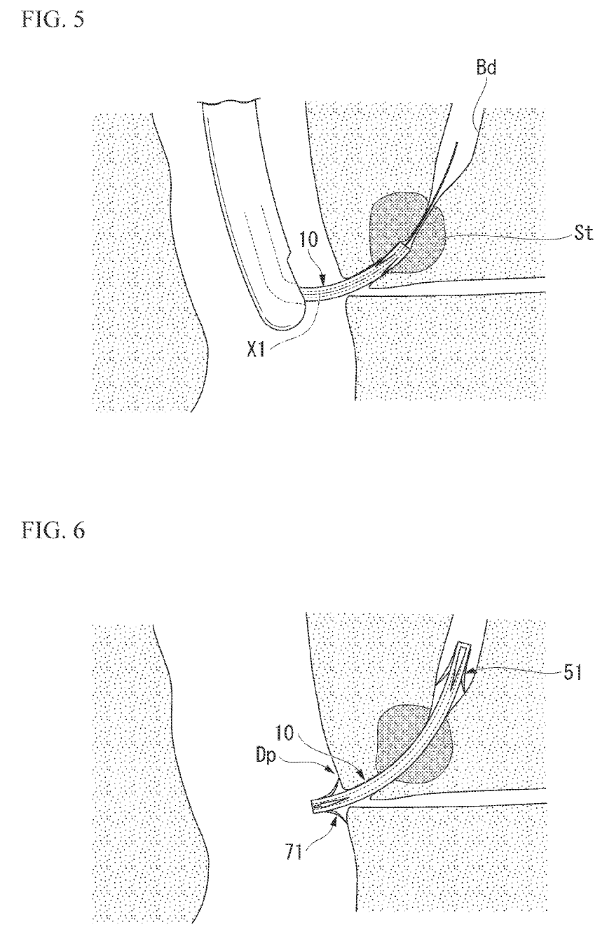

FIG. 5 is a diagram showing a process of placing the stent 10 using the stent delivery device 1. FIG. 6 is a diagram showing the placed stent 10.

First, a surgeon inserts a proximal end part of a guide wire protruding from a forceps port of an endoscope into a distal end opening of the tube 81 of the stent delivery device 1 to which the stent 10 is attached.

The guide wire enters the second lumen of the single lumen tube 91 through a proximal end opening of the tube 81. The surgeon causes the proximal end part of the guide wire to enter the guide wire lumen 92a, and the guide wire to protrude from a proximal end side opening of the guide wire lumen 92a.

The surgeon inserts the stent delivery device 1, through which the guide wire passes, into a channel of the endoscope such that a distal end of the stent delivery device 1 protrudes from a distal end of the channel. The surgeon operates a lifting base of the endoscope to point the distal end of the stent delivery device 1 toward a duodenal papilla and advances the stent delivery device 1 into a bile duct along the guide wire.

As shown in FIG. 5, the stent delivery device 1 advances into a bile duct Bd while it pushes and widens a stenosis site St in the bile duct Bd.

In this case, the stent 10 attached to the stent delivery device 1 advances into the bile duct Bd from the distal end side A1. As described above, in the stent 10, the first region 30a having a greater rigidity than the second region 30b disposed on the proximal end side A2 is disposed on the distal end side A1.

For that reason, the stent 10 can push and widen the stenosis site St and advance into the bile duct Bd while sufficiently maintaining a space in the bile duct Bd.

When the stent 10 is caused to enter the stenosis site St of the bile duct Bd, a force is applied to the stent 10 to compress the stent 10 in the longitudinal direction A.

The main body 11 of the stent 10 has the first region 30a, the second region 30b, and the third region 30c, and thus the above-mentioned force acts in a distributed manner on the boundary between the first region 30a and the second region 30b, and on the boundary between the second region 30b and the third region 30c. As a result, buckling of the stent 10 can be inhibited when the stent 10 is placed.

After the stent 10 is advanced into the bile duct Bd to a position at which the first flap 51 is disposed deeper (distal end side A1) than the stenosis site St, the surgeon advances and retracts the stent delivery device 1 to determine the placement position of the stent 10.

In this case, by checking the wire 30 provided in the first region 30a located between the distal end 12 of the main body 11 and the first fixed end 56 under X-ray fluoroscopy, the surgeon can confirm a distal end position of the stent 10 and determine the placement position of the stent 10.

In the stent delivery device 1, as long as the tube 81 is inserted through the loop of the thread 95, the stent 10 will not come off the pusher catheter 90, and thus the stent 10 can be pulled back by retracting the stent delivery device 1. For that reason, the surgeon can easily adjust the position of the stent 10.

After the placement position of the stent 10 is determined, the surgeon pulls the operation part 89 toward the surgeon side (proximal end side A2) while holding the pusher catheter 90. By pulling the operation part 89 toward the surgeon side, the wire 87 and the tube 81 retract. In this case, the stent 10 does not retract since it is in contact with the pusher catheter 90 in the longitudinal direction A.

When the tube 81 retracts and comes off the stent 10 and the thread 95, the engagement between the stent 10 and the pusher catheter 90 due to the thread 95 is released, and as shown in FIG. 6, the stent 10 is placed in a predetermined position in the bile duct Bd.

After the stent 10 is placed, as shown in FIG. 6, the first flap 51 is disposed on a deeper side of the stenosis site St, and the second flap 71 is disposed near a duodenal papilla Dp outside the bile duct Bd, and thus the placement position of the stent 10 is appropriately maintained.

In the stent 10 placed in the bile duct Bd, the second region 30b and the third region 30c having a smaller rigidity than the first region 30a are located on the proximal end side A2 of the first fixed end 56.

For that reason, the stent 10 is placed in the bile duct Bd while it sufficiently conforms to a shape of the bile duct Bd.

Next, an operation of retrieving the stent 10 placed in the bile duct Bd through the channel of the endoscope will be described.

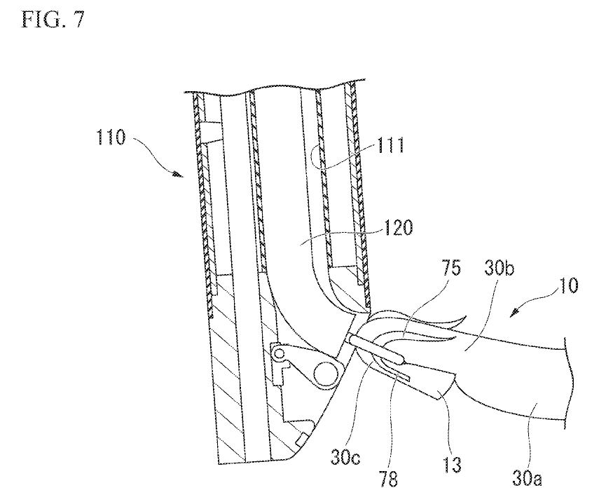

FIG. 7 is a diagram showing a process of pulling the stent 10 into the channel of the endoscope.

The surgeon introduces a side-viewing endoscope to the vicinity of the duodenal papilla Dp, causes a retrieval tool such as gripping forceps or a snare to protrude from a channel thereof, and holds the proximal end part of the stent 10 with the retrieval tool.

Next, the surgeon retracts the retrieval tool 120 holding the stent 10. As shown in FIG. 7, when the stent 10 reaches a channel distal end opening of the side-viewing endoscope (endoscope) 110, the stent 10 is pulled into a channel 111 of the endoscope 110 while it is folded back at a portion held by the retrieval tool 120.

In this case, the distance from the proximal end 13 of the main body 11 to the boundary between the second region 30b and the third region 30c (second distance L2) is twice or more the distance from the proximal end 13 of the main body 11 to the second fixed end 76 (first distance L1), and thus the surgeon can easily crush the stent 10 by folding back the third region 30c having the least rigidity, and pull it into the channel 111.

Also, as described above, the width of the rib 78 is smaller than the width of the second locking member 75. For that reason, a volume per unit length of the rib 78 is smaller than a volume per unit length of the second locking member 75. Thus, hindrance of the rib 78 to the crush of the third region 30c can be inhibited.

The medical stent (stent) 10 of the present embodiment includes the main body 11 including the tubular member (the inner layer 20 and the outer layer 40) that has a tubular shape and extends in the longitudinal direction A, and the reinforcing material (wire) 30 that is wound in a spiral shape around the longitudinal axis X1 extending in the longitudinal direction A to extend in the longitudinal direction A and embedded between the inner circumferential surface of the tubular member and the outer circumferential surface of the tubular member, the first locking member 55 that includes the first fixed end 56 fixed to the distal end side A1 of the main body 11 and the first free end 57 located on the outer side R2 from the outer circumferential surface of the tubular member, and extends from the first fixed end 56 to the first free end 57 toward the proximal end side A2, and the second locking member 75 that includes the second fixed end 76 fixed to the proximal end side A2 of the main body 11 and the second free end 77 located on the outer side R2 from the outer circumferential surface of the tubular member, and extending from the second fixed end 76 to the second free end 77 toward the distal end side A1, in which the main body 11 includes the first region 30a, the second region 30b, and the third region 30c that have different winding pitches of the reinforcing material 30 from one another.

The first region 30a is a region located between the distal end 12 of the main body 11 and the first fixed end 56 in the longitudinal direction A. The second region 30b is a region that is located on the proximal end side A2 of the first region 30a, and in which the reinforcing material 30 is wound in a spiral shape with a greater winding pitch than the first region 30a. The third region 30c is a region that is located on the proximal end side A2 of the second region 30b, and in which the reinforcing material 30 is wound in a spiral shape with a greater winding pitch than the second region 30b.

The stent delivery device 1 of the present embodiment includes the guide catheter 80 including the guide tube (tube) 81 through which the guide wire is configured to be inserted and the wire 87 connected to the guide tube 81, the pusher catheter 90 including the first tube (multi-lumen tube) 92 that includes the first lumen (wire lumen) 92b through which the wire 87 is configured to be inserted, and the second tube (single lumen tube) 91 that is connected to the distal end part of the first lumen 92b and includes the second lumen through which the guide tube 81 is configured to be inserted, the coupling member (thread) 95 that includes the loop through which the guide tube 81 is configured to be inserted and is coupled to the distal end part of the pusher catheter 90, and the stent 10 that extends in the longitudinal direction A and is located on the distal end side A1 from the distal end of the second tube 91.

The main body 11 includes the through hole (second through hole) 15 that penetrates the tubular member from the inner circumferential surface to the outer circumferential surface and is provided on the proximal end side A2. The loop of the coupling member 95 is inserted into the through hole 15.

By inserting the guide tube 81 through the loop of the coupling member 95, the stent 10 is positioned relative to the pusher catheter 90.

Accordingly, according to the medical stent 10 and the stent delivery device 1 of the present embodiment, it is possible to provide the medical stent 10 and the stent delivery device 1 in which the position of the distal end 12 can be easily checked under X-ray fluoroscopy.

Although one embodiment of the present invention has been described with reference to the drawings, a specific configuration thereof is not limited to the present embodiment, and design changes or the like that do not deviate from the spirit of the present invention are also included. Further, the constituent elements shown in the above-described embodiment and the modified examples shown below can be appropriately combined together to configure the present invention.

### (Modified example 1)

In the above embodiment, the proximal end of the third region 30c is located at the proximal end 13 of the main body 11, but a form of the third region 30c is not limited thereto. The proximal end of the third region 30c may be located, for example, closer to the distal end side A1 than the proximal end 13 of the main body 11.

In this case, the main body of the stent is provided with, in order from the distal end side A1, the first region 30a, the second region 30b, the third region 30c, and a region in which the wire 30 is not provided (fourth region).

The rigidity of the fourth region in which the wire 30 is not provided is smaller than the rigidity of the third region 30c. That is, the main body of the stent includes four regions with different rigidities.

The main body of the stent includes the first region 30a, the second region 30b, the third region 30c, and the fourth region described above, and thus the force acting on the stent when the stent is advanced into the stenosis site St of the bile duct Bd acts in a distributed manner on the boundary between the first region 30a and the second region 30b, the boundary between the second region 30b and the third region 30c, and the boundary between the third region 30c and the fourth region. As a result, buckling of the stent can be appropriately inhibited when the stent is placed.

In this case, for example, at least a part of the rib 78 is located between the proximal end of the third region 30c and the proximal end 13 of the main body 11 in the longitudinal direction A. That is, since the rib 78 is provided in the fourth region in which the wire 30 is not provided, the rib 78 can provide a predetermined rigidity.

### (Modified example 2)

In the above embodiment, the main body 11 has the same outer diameter from the distal end 12 to the proximal end 13, but a form of the main body 11 is not limited thereto.

For example, the main body of the stent may have a narrowed diameter part at the distal end part, in which a diameter of the outer circumferential surface is narrowed toward the distal end side A1.

The narrowed diameter part is provided, for example, on the distal end side A1 from the first fixed end 56. In this narrowed diameter part, a diameter of the inner circumferential surface of the main body of the stent may be narrowed toward the distal end side A1, or a diameter of the wire (reinforcing material) embedded in the main body may be narrowed toward the distal end side A1.

The distal end of the stent is tapered due to the narrowed diameter part, and thus when the stent is advanced into the bile duct Bd, the distal end of the stent can easily be advanced into the stenosis site St.

While preferred embodiments of the invention have been described and illustrated above, it should be understood that these are exemplary examples of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

What is claimed is:

1. A medical stent comprising:
a main body including a tubular member that has a tubular shape and extends in a longitudinal direction thereof, and a reinforcing material that is wound in a spiral shape around a longitudinal axis extending in the longitudinal direction and embedded between an inner circumferential surface of the tubular member and an outer circumferential surface of the tubular member; and
a first locking member that includes a first fixed end fixed to a distal end side in the longitudinal direction of the main body and a first free end located on an outer side in a radial direction of the main body from the outer circumferential surface of the tubular member,
wherein the main body includes a first region, a second region, and a third region that have different winding pitches of the reinforcing material from one another,
the first region is a region located between the distal end of the main body and the first fixed end in the longitudinal direction,
the second region is a region that is located on a proximal end side of the first region and in which the reinforcing material is wound in a spiral shape with a greater winding pitch than the first region, and
the third region is a region that is located on the proximal end side of the second region and in which the reinforcing material is wound in a spiral shape with a greater winding pitch than the second region.

2. The medical stent according to claim 1,
wherein the first locking member extends from the first fixed end to the first free end toward the proximal end side in the longitudinal direction.

3. The medical stent according to claim 1,
wherein a second locking member that includes a second fixed end fixed to the proximal end side of the main body and a second free end located on the outer side from the outer circumferential surface of the tubular member, and extends from the second fixed end to the second free end toward the distal end side.

4. The medical stent according to claim 1,
wherein a boundary between the first region and the second region is provided at substantially the same position as the first fixed end in the longitudinal direction.

5. The medical stent according to claim 3,
wherein a distance from a boundary between the second region and the third region to a proximal end of the main body in the longitudinal direction is at least twice a distance from the second fixed end to the proximal end of the main body.

6. The medical stent according to claim 1,
wherein a proximal end of the third region is located at a proximal end of the main body.

7. The medical stent according to claim 1,
wherein a proximal end of the third region is located closer to the distal end side than a proximal end of the main body.

8. The medical stent according to claim 3, further comprising:
a joint part that extends from the second fixed end toward the proximal end side and is joined to the outer circumferential surface of the main body.

9. The medical stent according to claim 3,
wherein a proximal end of the third region is located closer to the distal end side than a proximal end of the main body,
the medical stent further comprising a joint part that extends from the second fixed end toward the proximal end side and is joined to the outer circumferential surface of the main body, and
at least a part of the joint part is located between the proximal end of the third region and the proximal end of the main body in the longitudinal direction.

10. The medical stent according to claim 1, further comprising:
a through hole penetrating the main body from the inner circumferential surface to the outer circumferential surface,
wherein the through hole is provided to overlap the first region in the longitudinal direction.

11. The medical stent according to claim 1, further comprising:
an annular ring with the longitudinal axis set as its central axis,
wherein the ring is provided at a distal end of the first region and fixes the reinforcing material to the main body.

12. The medical stent according to claim 1,
wherein the main body includes a narrowed diameter part at its distal end, whose diameter is narrowed from the outer circumferential surface toward the distal end side.

13. A stent delivery device comprising:
a guide catheter including a guide tube through which a guide wire is configured to be inserted, and a wire connected to the guide tube;
a pusher catheter including a first tube that includes a first lumen through which the wire is configured to be inserted, and a second tube that is connected to a distal end part of the first lumen and includes a second lumen through which the guide tube is configured to be inserted;
a coupling member that includes a loop through which the guide tube is configured to be inserted and is coupled to the distal end part of the pusher catheter; and
a stent that extends in a longitudinal direction thereof and is located on a distal end side in the longitudinal direction from a distal end of the second tube,
the stent including:
a main body including a tubular member having a tubular shape, which extends in the longitudinal direction and through which the guide wire is inserted, a reinforcing material that is wound in a spiral shape around a longitudinal axis extending in the longitudinal direction to extend in the longitudinal direction and embedded between an inner circumferential surface of the tubular member and an outer circumferential surface of the tubular member, and a through hole that penetrates the tubular member from the inner circumferential surface to the outer circumferential surface and is provided on a proximal end side in the longitudinal direction;
a first locking member that includes a first fixed end fixed to the distal end side of the main body and a first free end located on an outer side in a radial direction of the main body from the outer circumferential surface of the tubular member, and extends from the first fixed end to the first free end toward the proximal end side; and
a second locking member that includes a second fixed end fixed to the proximal end side of the main body and a second free end located on the outer side from the outer circumferential surface of the tubular member, and extends from the second fixed end to the second free end toward the distal end side,
wherein the main body includes a first region, a second region, and a third region that have different winding pitches of the reinforcing material from one another,
the first region is a region located between the distal end of the main body and the first fixed end in the longitudinal direction,
the second region is a region that is located on the proximal end side of the first region, and in which the reinforcing material is wound in a spiral shape with a greater winding pitch than the first region,
the third region is a region that is located on the proximal end side of the second region, and in which the reinforcing material is wound in a spiral shape with a greater winding pitch than the second region,
the loop of the coupling member is inserted into the through hole, and
the guide tube is inserted into the loop, thereby positioning the stent relative to the pusher catheter.
